(19) United States
(12) Patent Application Publication
(10) Pub. No.: US 2025/0000345 A1
(43) Pub. Date: Jan. 2, 2025

(54) AN ENDOSCOPE

(71) Applicant: AMBU A/S, Ballerup (DK)

(72) Inventor: Victor SILBERMANN, Them (DK)

(73) Assignee: AMBU A/S, Ballerup (DK)

(21) Appl. No.: 18/693,254

(22) PCT Filed: Sep. 22, 2022
(86) PCT No.: PCT/EP2022/076404

§ 371 Date: Mar. 19, 2024

(30) Foreign Application Priority Data
Sep. 28, 2021 (DK) PA 2021 70474

(51) Int. Cl. A61B 1/005; A61B 1/00

(52) U.S. Cl. CPC A61B 1/0052; A61B 1/0004; A61B 1/00045

(57) ABSTRACT

An endoscope (1) including a proximal handle (5). The handle includes (5) at least one handle housing part (9) having an interior surface and an exterior surface. The handle housing part includes a recess forming part of the exterior surface. An electrical switch is accommodated on the exterior surface in said recess and is digitally operable via an operating member (15). The handle housing part (9) includes a through hole provided in said recess and interconnecting the exterior surface and the interior surface and the operating member includes at least one retaining member passing through the through hole in the handle housing part (9) from said recess and engages the interior surface.

The present disclosure relates to endoscopes in particular but not exclusively to disposable video endoscopes.

In general, an endoscope comprises an operating handle at the proximal end and an insertion cord extending from the handle towards the distal end. The handle is adapted to be held by an operator and inter alia comprises externally protruding operating members connected to internal control gear allowing the operator to control the movement of a controllable bending section at the distal end of the insertion cord, while advancing the distal end of the insertion cord to a desired location e.g. within a body cavity of a person.

The controllable bending section is normally an articulated section at the distal tip of the insertion cord that can be controlled by the operator from the handle, allowing the operator to advance the distal tip of the endoscope to a desired location of interest by means of a series of action involving inter alia bending the bending section in a desired direction, advancing the insertion cord and turning the insertion cord by turning the handle which is rigidly connected thereto. During this insertion process, the operator finds his way by means of an attached monitoring device, such as a monitor with a display screen allowing the operator to follow his or her actions in real time as capture by the electronic imaging located in or at the distal tip of the insertion cord of the endoscope.

During this insertion process or once the tip of the endoscope has arrived at the location of interest there may be a need for image capture from the electronic imaging device and/or activation of other functions that may be electrically controlled, as well as other mechanically effected procedures.

For this it is known to provide electrical switches at the handle of the endoscope, e.g. at a location easily reachable by the index finger of the hand with which the operator holds the endoscope. Examples of such electrical switches are inter alia found in patent application JP-9-173278 and patent JP3631314.

Common to JP-9-173278 and JP3631314 is that the switches are arranged deep within the handle and actuated via relatively long push-button stems from the push-buttons on the exterior of the handle. These arrangements are rather complicated and do not lend themselves to single use endoscopes where parts must be kept simple and easy to assemble. Moreover, because space is sparse within the handle, the handles may need to be made larger, more bulky and less ergonomically suitable than desired.

According to a first aspect of the present disclosure these drawbacks are overcome by an endoscope comprising a proximal handle and an insertion cord where the handle comprises at least on handle housing part having an interior surface and an exterior surface and the insertion cord comprises a camera arranged at the distal end of the insertion cord, said handle housing part comprises a recess forming part of the exterior surface, at least one electrical switch digitally operable via an operating member associated with said electrical switch, said switch being accommodated on the exterior surface in said recess, said handle housing part comprises a through hole provided in said recess and interconnecting the exterior surface and the interior surface, said operating member comprising at least one retaining member passing through the through hole in the handle housing part from said recess and engaging the interior surface. Placing the switches on the exterior solves the problems with lack of space within the handle. This is inter alia possible because in the prior art endoscopes the switches needed to be rugged and protected, i.e. within the handle, because they needed to be able to withstand the harsh procedure of cleaning and sterilisation such as in an autoclave.

According to a second aspect of the disclosure these drawbacks are overcome by a system comprising a display device and an endoscope according to the first aspect connectable to the display device.

According to an embodiment of the first aspect of the disclosure, said at least one retaining member comprises two legs each leg being provided with a barb comprising a camming surface and a latching surface. This provides a single operating member which is not only easy to position and assemble with the remainder of the endoscope handle, but which may also provide essentially the only moving part of the switch apart from the electrical contacts.

According to an embodiment according to the first aspect of the disclosure, said camming surfaces face away from a centre plane of said operating member in mutually opposite directions. This allows for precise sideways location of the operating member an provides symmetric deflection to both sides if the operating member is to operate two switches.

According to another embodiment according to the first aspect of the disclosure, the cross-sectional area of the through hole is smaller at the interior surface than at the exterior surface. This further facilitates the precise sideways location of the operating member by providing surfaces over which the camming surface of the slide before the latching surface engages the interior wall.

According to a preferred embodiment of the first aspect of the disclosure, the at least one electrical switch is comprised in a foil switch. Foil switches as advantageous in that they are flat and therefore may be located on the outside of the handle, e.g. in a shallow recess in the exterior surface, so as to be easily reachable with the index finger for the operator. Moreover, such foil switches may be sealed as they comprise multiple layers of metal and polymer materials laminated together over most of the area, thus not allowing ingress of water etc. to the contacts.

According to an embodiment of the first aspect of the disclosure, the foil switch is adhered to the exterior surface in said recess. This is easy during assembly and allows the switch to be secured in the correct position with respect to the operating member.

According to an embodiment of the first aspect of the invention the operating member comprises at least one protrusion adapted to activate said at least one electrical switch. Thus, correct depressing of the switch will be ensured because the switch is maintained in correct position by the adhesive, and the protrusion is aligned therewith because of the central arrangement of the legs.

An endoscope according to any one of the preceding claims wherein the exterior surface comprises one or more protrusions and/or recesses adapted to serve as a fulcrum for pivotal motion of the operating member. This provides an axis for the operating member to pivot about and further ensures the correct central location of the operating member. Furthermore, together with the latching surfaces of the legs it ensures that the operating member is held in engagement with the handle housing with a minimum of play.

According to an embodiment of the first aspect of the disclosure, the operating member comprises at least one protrusion adapted to activate said at least one electrical switch. This provides a precise pressing point on the switch. This is especially relevant if the switch is a foil switch, be it with or without an internal dome spring.

According to an embodiment of the invention, the recess comprises at least one protrusion adapted to aid in the positioning of the at least one electrical switch. A protrusion in conjunction with an edge or a corresponding cut-out in the foil switch will ensure easy and correct positioning of the foil switch in the recess during assembly.

The disclosure will now be made in greater detail based on nonlimiting exemplary embodiment and with reference to the drawings on which:

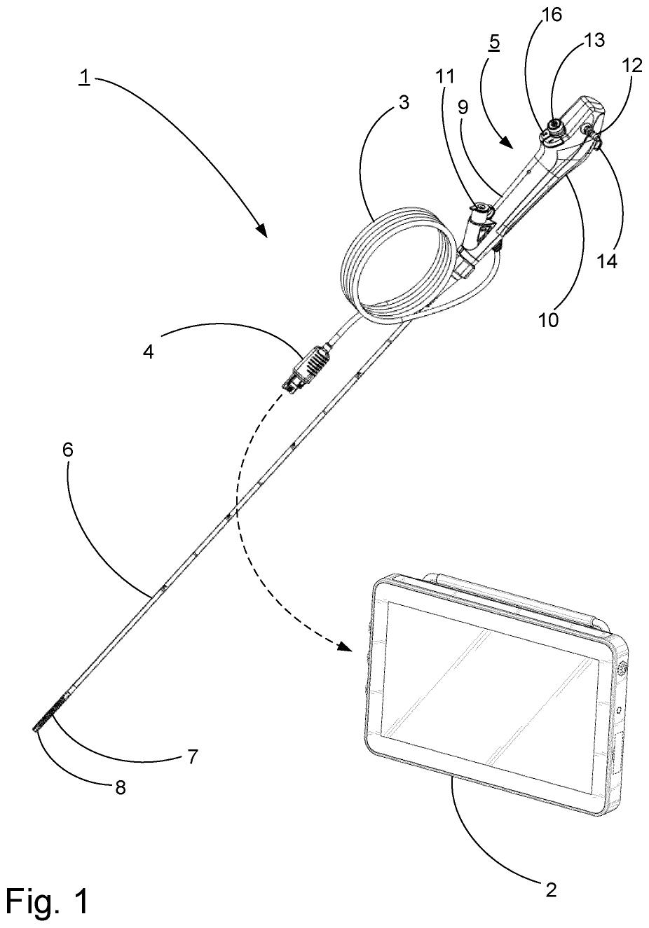

FIG. 1 shows a system comprising a display device and an endoscope according to the disclosure,

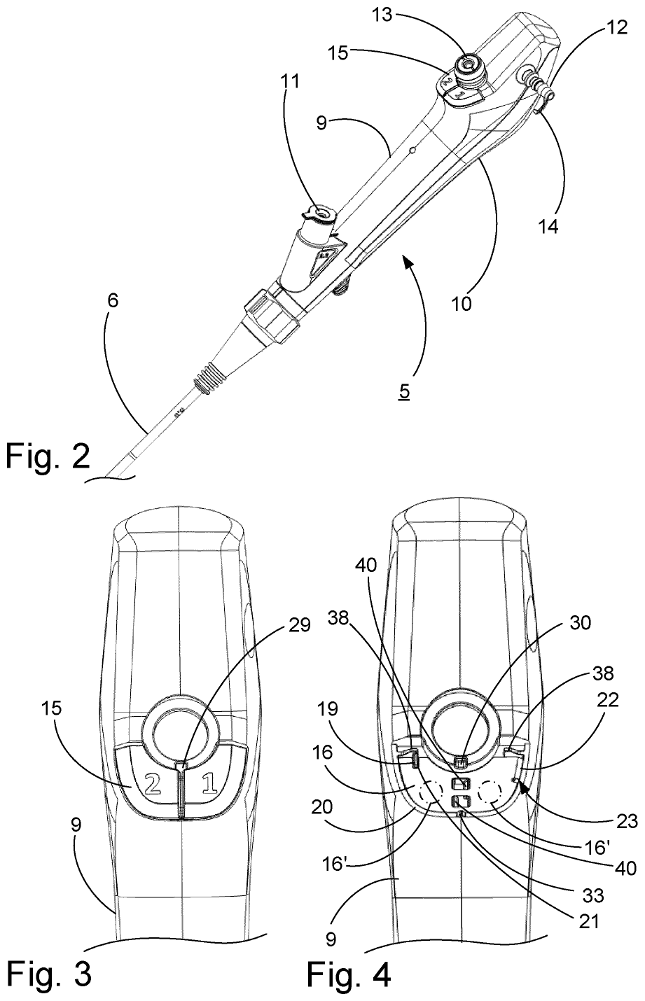

FIG. 2 shows the front of the handle in isometric view,

FIG. 3 shows a front view of the upper part of the handle where two electrical switches are located,

FIG. 4 shows a front view corresponding to that of FIG. 3 with the operating member removed,

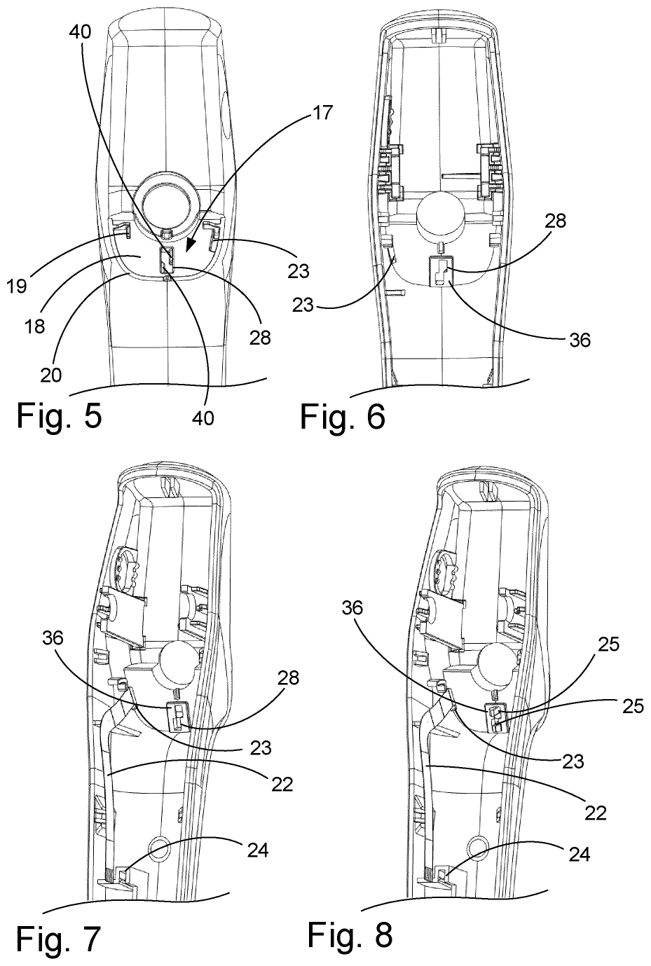

FIG. 5 shows a front view corresponding to that of FIGS. 3 and 4 with also the switches removed,

FIG. 6 shows an interior view from the back of the upper part of the handle housing part only,

FIG. 7 shows a slightly angled interior view of the upper part of the handle housing with the switch mounted,

FIG. 8 shows an interior view corresponding to that of FIG. 7 but with the operating member also mounted,

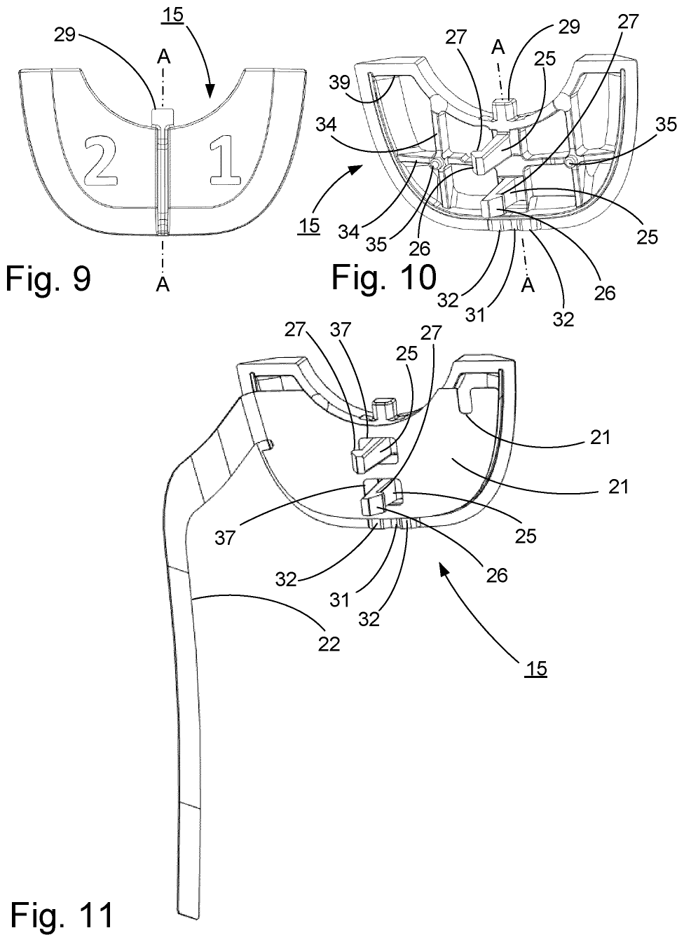

FIG. 9 shows a front view of the operating member,

FIG. 10 shows a slightly angled rear view of the operating member, and

FIG. 11 shows a view of the operating member corresponding to that of FIG. 10 but with the switch foil mounted.

Turning first to FIG. 1 an endoscope 1 and a display device 2 are shown. The endoscope 1 is connectable to the display device 2 via a connection such as a cable 3 with a connector 4 as illustrated with the dashed arrow to form an endoscope system.

The endoscope 1 comprises a proximal handle 5 and an insertion cord 6 with a bending section 7 and a distal tip part 8 in which an electronic imaging device, e.g. a camera (not visible) is accommodated. The images recorded by the camera may be communicated to and displayed on the display device 2 in real time for the operator to follow his or her actions when using the endoscope 1.

The handle 5 comprises at least one handle housing part in the illustrated embodiment a front handle housing part 9 as well as a second handle housing part, i.e. a rear handle housing part 10. The handle 5 furthermore may comprise features useful in endoscopy, such as a tool insertion port 11 for insertion tools through a working channel in the insertion cord 6, a suction connector 12 for connecting a suction channel or working channel of the endoscope to a vacuum source, a valve operating button 13 for activating said suction an a control knob for lever 14 for controlling the bending section 7. The control knob or lever 14 will normally be located at a position where it is easily accessible by the thumb of the hand with which the operator grips the handle 5. The valve operating button 13 will normally be located at a location where it is easily accessible by the index finger of the hand with which the operator grips the handle 5.

In accordance with the disclosure the handle 5 furthermore comprises one or more switches 16′ (indicated with interrupted lines in FIG. 4) operable by means of a switch operating member 15. In the illustrated embodiment there are two switches 16′ provided as two sets of contacts (not visible) integrated in a single laminated switch foil 16. Foil switches are advantageous in this context as they may be provided as part of a flat, flexible, sealed switch foil 16. The switch foil 16 may be pre-fabricated as self-adhesive foil that may be attached in the predetermined location upon removal of a release liner. This makes it easy to place during assembly of the endoscope. Being flat, the switch foil 16 may be accommodated in a shallow recess 17 provided as a part of the exterior surface of the at least one handle part, in case the front handle part 9.

In order to place the switch foil 16 in correct position in the bottom 18 of the recess 17 one or more protrusions 19 protruding from the bottom of the recess may be provided. Evidently the rim 20 of the recess will also aid in positioning the switch foil 16 correctly if it has a matching shape in addition to cut-outs 21 matching the protrusions 19. This positioning will of course work irrespectively of whether the switch foil 16 is just held loosely in the recess 17 or adhered to the bottom 18 of the recess 17. There may be additional walls 38 provided in the recess 17 to aid in positioning the switch foil 16, and serve as guides for the operating member 15 in order to avoid undesired twisting motion, e.g. by overlapping parts of a circumferential wall 39 of the operating member 15.

The switch foil 16 preferably has a flat integrated connecting cable 22 with internal wires. The flat integrated connecting cable 22 passes through a suitable wire passage 23 in the handle part 9 into the handle where it may be connected to a printed circuit board (not shown), e.g. accommodated in a suitable receptacle 24 provided as part for the interior housing surface.

During assembly, after the switch foil 16 is in place in the recess 17 and the integrated connecting cable 22 has been passed through the passage 23 to the interior side of the front handle part 9 the switch operating member 15 is placed over the switch foil 16, except for a pair of legs 25 that pass through suitable apertures 37, cf. FIG. 11, in the switch foil 16, and in engagement with the front handle part 9 to complete the switch.

For this engagement the operating member 15 comprises the pair of protruding legs 25 which extend perpendicularly to from the rear side of the operating member 15, the front side being understood as the one digitally engaged by the index finger of the operator. As can best be seen in FIGS. 10 and 11 these legs each comprise a camming surface 26 and an undercut latching surface 27 to form a barb allowing them to latch the interior surface of the front handle part 9 inside the handle 5, when they have passed through a corresponding through hole 28 in the front housing part 9 provided in the bottom 18 of the recess 17 and interconnecting the exterior surface and the interior surface. As can be seen, the though hole 28 may be surrounded by a raised area 36 where the wall thickness between the bottom of the recess 18 and the interior surface is increased for improved strength and stability.

As can be seen from comparison between FIGS. 5 and 6, the cross-sectional area of the through hole is smaller at the interior surface than at the exterior surface. At the exterior surface in the bottom 18 of the recess 17 the cross-section is a rectangle, whereas on the interior the cross-section is more like are two smaller rectangles with a mutual off-set on either side of a centre line aligned with the centre line A-A, which also defines a pivot axis for the operating member. The area of the two smaller rectangles in the interior surface together is smaller the area of rectangle in the bottom 18 of the recess 17. Thus two ramps 40 are formed which will be engaged by respective camming surfaces 26 to deflect the legs during insertion and allowing the legs to snap back and place the undercut latching surfaces 27 in engagement with the interior surface of the front handle housing 9. Both the ramps 40 and the camming surfaces 26 face in opposite directions with respect to the centre line of the through hole 28 and the pivot axis A-A, so that the alignment between the centre line of the through hole 28 and the pivot axis A-A in ensured. This geometry also ensures that the legs can bend sideways in the through hole 28 away from the centre axis when the operating member 15 pivots about the pivot axis A-A.

The length of the 25 legs to the latching surfaces 27 are so dimensioned that when the barbs snap into engagement, the operating member is securely retained in engagement in a pair of bearings aligned in parallel with the centre line of the through hole 28, thus also ensuring the alignment of the pivot axis A-A for the pivotal movement of the operating member 15. As the skilled person will know, such bearings may be devised in various ways. It is currently preferred that one bearing is a trunnion type bearing whereas the other comprises a fulcrum.

Thus, as can best be seen from FIGS. 9-11, the operating member comprise an axially protruding trunnion 29. The trunnion 29 may be semicircular, circular, rounded or, as currently preferred, have a somewhat trapezoid cross-section with rounded corners.

The trunnion 29 rests in a somewhat larger, but likewise trapezoid bearing recess 30 formed in the exterior surface of the front housing part 9.

The reasons for currently preferring a trunnion 29 with a trapezoid cross-section with rounded corners are two. First because it provides a larger contact surface in the rest position in the bearing recess 30. In terms of molding, the trapezoid shape also provides a more optimal geometry for injection molding compared to a circular shape. This also applies to the bearing recess.

The other bearing is provided by a gap 31 between a pair of protrusions 32 on the rear side of the operating member 15. The gap 31 rests on a trapezoid or triangular fulcrum protrusion 33. Other shapes are evidently also possible, e.g. protrusion or at least the top of it could be rounded. However, for the same general reasons as for the trunnion 29 and recess 30, trapezoidal shapes are currently preferred for both the fulcrum 33 and the gap 31.

The switch operating member 15 is preferably made by injection moulding with reinforcement ribs 34 arranged on the rear side, where they are not visible after assembly. This keep the material consumption and cycle time during moulding down. The reinforcement ribs 34 are preferably arranged in a cross-wise pattern. Preferably, where the arms of the crosses meet, the switch operating member 15 comprises at least one protrusion 35 adapted by height, shape and position to activate said at least one electrical switch 16′. That is to say they match the location of the electrical contacts of the switches 16′ hidden inside the switch foil 16. The electrical contacts may comprise metal domes or other means for biasing the contacts away from each other when the switch 16′ is not activated. Switches 16′ in switch foils 16 are per se known and come in numerous variants and may largely be a matter of selection for the skilled person. The switch foil 16 currently preferred, however, has two conductors for each switch 16 so as to have the grounded conductor largely surrounding the other, in order to protect the signal conductors against electrical strikes, e.g. from static electricity. Similarly, there will be four wires in the flat connecting cable 22, arranged so that the ground wires run along the edges and the signal wires run centrally between the ground wires.

The present invention has now been given based on exemplary embodiments. The skilled person will however know that numerous variants are possible without deviating from the scope of the invention as defined in the claims. Thus, the pivoting operating member 16 need not be associated with more than a single switch. Other switch types than foil switches 16′ will also be possible, as long as the overall height does not disturb the ergonomics of the handle. The switch need not form part of the front handle part.

We claim:

1. An endoscope comprising a proximal handle and an insertion cord where the handle comprises at least one handle housing part having an interior surface and an exterior surface and the insertion tube comprises a camera arranged at the distal end of the insertion cord,
said handle housing part comprises a recess forming part of the exterior surface,
at least one electrical switch digitally operable via an operating member associated with said electrical switch,
said switch being accommodated on the exterior surface in said recess,
said handle housing part comprises a through hole provided in said recess and interconnecting the exterior surface and the interior surface,
said operating member comprising at least one retaining member passing through the through hole in the handle housing part from said recess and engaging the interior surface.

2. The endoscope of claim 1, wherein said at least one retaining member comprises two legs, each leg comprising a barb comprising a camming surface and a latching surface.

3. The endoscope of claim 2, wherein said camming surfaces faces away from a centre plane of said operating member in mutually opposite directions.

4. The endoscope of claim 3, wherein a cross-sectional area of the through hole is smaller at the interior surface than at the exterior surface.

5. The endoscope of claim 1, wherein the at least one electrical switch is comprised in a foil switch.

6. The endoscope of claim 5, wherein the foil switch is adhered to the exterior surface in said recess.

7. The endoscope of claim 1, wherein the operating member comprises at least one protrusion configured to activate said at least one electrical switch.

8. The endoscope of claim 1, wherein the exterior surface comprises one or more protrusions and/or recesses configured to serve as a fulcrum for pivotal motion of the operating member.

9. The endoscope of claim 1, wherein said recess comprises at least one protrusion configured to aid in the positioning of the at least one electrical switch.

10. A system comprising a display device and the endoscope of claim 1, the endoscope being connectable to the display device.

11. The endoscope of claim 1, wherein said at least one retaining member comprises two legs, each leg comprising a barb comprising a camming surface and a latching surface, wherein the camming surfaces face away from each other in mutually opposite directions.

12. The endoscope of claim 1, wherein the at least one electrical switch is comprised in a foil switch adhered to the exterior surface in said recess.
